(19) United States
(12) Patent Application Publication
(10) Pub. No.: US 2013/0317095 A1
(43) Pub. Date: Nov. 28, 2013

(54) Use of Artemisine Derivatives and Pharmaceutical Salts Thereof

(71) Applicants: Jianqing Mi, Shanghai (CN); Ying Li, Shanghai (CN); Yu Peng, Shanghai (CN); Yu Zhang, Shanghai (CN); Ruimin Nie, Shanghai (CN); Jingjing Liu, Shanghai (CN); Jin Wang, Shanghai (CN); Yueying Wang, Shanghai (CN); Xun Cai, Shanghai (CN); Yang Li, Shanghai (CN); Saijuan Chen, Shanghai (CN); Zhenyi Wang, Shanghai (CN)

(72) Inventors: Jianqing Mi, Shanghai (CN); Ying Li, Shanghai (CN); Yu Peng, Shanghai (CN); Yu Zhang, Shanghai (CN); Ruimin Nie, Shanghai (CN); Jingjing Liu, Shanghai (CN); Jin Wang, Shanghai (CN); Yueying Wang, Shanghai (CN); Xun Cai, Shanghai (CN); Yang Li, Shanghai (CN); Saijuan Chen, Shanghai (CN); Zhenyi Wang, Shanghai (CN)

(73) Assignee: Shanghai Institute of Materia Medica, Chinese Academy of Sciences, Shanghai (CN)

(21) Appl. No.: 13/982,684

(22) PCT Filed: Jan. 16, 2012
(86) PCT No.: PCT/CN2012/070401

§ 371 Date: Jul. 30, 2013

(30) Foreign Application Priority Data
Jan. 31, 2011 (CN) 201110034201.X

(51) Int. Cl. C07D 493/18; C07C 57/145

(52) U.S. Cl. CPC C07D 493/18; C07C 57/145
USPC 514/450; 549/348

(57) ABSTRACT

The invention demonstrates the application of an artemisinin derivative and its pharmaceutical salt. The artemisinin derivatives diarteether amine and its pharmaceutical salt inhibit the proliferation of leukemic cells, block the cell cycle of leukemic cells and induce the apoptosis of leukemic cells. Artemisinin derivatives of the present invention and its pharmaceutical salt can be used for the preparation of anti-leukemia medicines, especially for treatment of acute leukemia and, what's more, for the treatment of acute myeloid leukemia.

### FIELD OF THE INVENTION

This invention belongs to medical field, specifically, relating to the application of a kind of artemisinin-derivatives and its pharmaceutical salts.

### BACKGROUND OF THIS INVENTION

Leukemia, a group of diseases characterized by the malignant cloning of the HSCs, is one of the malignant tumors of hematologic system, which jeopardizes the health of human beings. Acute leukemia, especially, is a type of rapidly progressing diseases, which lead to the accumulation of a sum of immature blood cells in bone marrow and blood. It can be divided into acute nonlymphocytic leukemia(ANLL) and acut lymphocytic leukemia(ALL).

Acute myelocytic leukemia(AML) or acute nonlymphocytic leukemia(ANLL) includes all the acute leukemia derived from nonlymphocytes. AML is a group of diseases attributing to mutation of karyotype in pleuripotent stem cells or slightly differentiated precursor cells, which is a maligent cloning disease of hemopoietic system. The WHO classifications of Acute myelogenous leukemia (AML) are as follows: 1. AML with recurrent genetic abnormalities,including (1)AML with t(8;22)(q22;q22)(AML/ETO), characterized by the translocations between chromosome 8 and 21 [t(8;21)] and AML/ETO fusion genes; (2)AML with bone marrow eosinophilia inv(16) (p13;q22)or t(16;16)(p13;q22); (CBFβ/MYH11);(3)AML with t(15;17)(q22;q21),(PML-RARα) and its mution; (4) AML with abnormlity of 11q23(MLL); 2. AML with multilineage dysplasia; 3. AML and MDS, therapy-related; 4. AML not otherwise categorized, the definitions of most subtypes are same as the FAB classifications. The diagnostic standards are based on the mainly involved cell lineage and its degree of maturation (1) Acute myeloblastic leukemia, minimally differentiated (AML-M0 in FAB): under the light microscope, blasts are reminiscent of L2 cell, with distinct nucleolus, agranular cytoplasm, basophilia, and without azurophilic granule or Auer rods. Myeloperoxidase (MPO) and Sudan Black (SBB)<3%. On the electron microscope, MPO(+), myeloid markers such as CD33 or CD13 are positive. Lymphoid antigens, usually, are negative, but sometimes CD7+ and TdT+; (2) Acute myeloblastic leukemia without maturation (AML-M1 in FAB): Undifferentiated myeloblast (I+II)≧90% of non-erythroid cells in bone marrow, and at least 3% MPO(+) cells; (3) acute myeloblastic leukemia with maturation(AML-M2 in FAB): myeloblast (I+II)≧30% ˜89%of non-erythroid cells in bone marrow, monocytic elements <20% of non-erythroid cells, granulocytic elements at least 10% of non-erythroid cells. (4) acute myelomonocytic leukemia (AML-M4 in FAB): blasts≧30% of non-erythroid cells in bone marrow, granulocytic elements accounting for 30%˜80% of non-erythroid cells, monocytic cells covering at least 20% of cells in bone marrow, and CD14(+); (5) acute monocytic leukemia (AML-M5 in FAB): monoblasts and promonocytes≧30% of non-erythroid cells in bone marrow, CD14(+); (6) acute erythroid leukemia(AML-M6 in FAB):_erythroid precursors >50% of nucleated cells in bone marrow, blasts(I+II)≧30% of non-erythroid cells in bone marrow; (7) acute megakaryoblastic leukemia (AML-M7 in FAB): megakaryoblasts ≧30% of non-erythroid cells in bone marrow, CD41(+), CD61(+), and CD42(+).

Nowadays, the standard treatments to leukemia include regular chemotherapy, bone marrow transplantation and radiotherapy. However, the prognoses of the majorities are still bad. The survival rate is significantly affected by the severe side effect such as infections, haemorrhages, and rejections after the transplantation and the relapse. Thus it is vital to find the novel, efficient, anti-tumor drugs to enhance the rate of complete remission and the survival time. What's more, treatments and prognoses vary with diagnostic criteria and subtypes of leukemia. Therefore, it's necessary to diagnose specifically the type and subtype of the disease according to the features of the leukemic cells in morphology, immunology and cytogenetics.

### SUMMARY OF THE INVENTION

The object of the present invention is to provide a kind of artemisinin derivatives diarteethe amine and the application of its pharmaceutical salt.

The artemisinin derivative in this invention is diarteethe amine, and the structural formular is as follow:

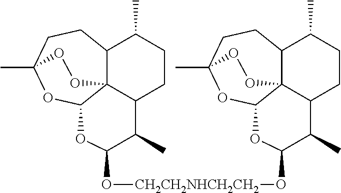

With extensive and intensive study, we find that hydrosoluble artemisinin derivative diarteethe amine maleate (marked with SM1044) can inhibit the proliferation of the leukemic cells, arrest cell cycle, and induce apoptosis.

So artemisinin derivative diarteethe amine and its pharmaceutical salt can be used to prepare the drugs that treat leukemia, especially the drugs aiming at acute leukemia, and more particularly, drugs aiming at acute myelocytic leukemia.

According to selected embodiments of the invention, the acute myelocytic leukemia as mentioned above includes AML with t(8;22)(q22;q22)(AML/ETO) and AML with t(15;17)(q22;q21),(PML-RARα) in AML with recurrent genetic abnormalities, and AML-M2 and AML-M5 in AML not otherwise categorized.

### DESCRIPTION OF FIGURES

FIG. 1: the inhibitory curve of AML cells treated with SM1044.(a) kasumi-1 cell; (b) NB4-R1 cell; (c) HL60 cell;(d)U937 cell.

FIG. 2:flow cytometry analysis of apoptosis rates of the AML cells treated with SM1044.(a) kasumi-1 cell; (b) NB4-R1 cell; (c) HL60 cell; (d) U937 cell.

FIG. 3: flow cytometry analysis of mitochondrial membrane potential of the AML cells treated with SM1044.(a) kasumi-1 cell; (b) NB4-R1 cell; (c) HL60 cell; (d) U937 cell.

FIG. 4: western blot analysis of apoptosis-related protein in the AML cells treated with SM1044.(1) NB4-R1 cell; (b) HL60 cell; (c) U937 cell.

FIG. 5A: degradation induced by SM1044 of the Kasumi-1 specific AML1-ETO fusion protein; FIG. 5B: the electrophoretogram analysis of the variation induced by SM1044 of Kasumi-1 specific AML1-ETO fusion gene in the level of mRNA.

FIG. 6: western blot analysis of the protein expression of c-myc (an oncogene over-expressed in HL60 cells) in HL60 cells treated with SM1044.

FIG. 7: SM1044 blocking the cell cycle of AML cells, (a) Kasumi-1 cells; (b) HL60 cells.

FIG. 8: the growth curve of transplanted tumor with AML cells, (a)kasumi-1 cells; (b) HL60 cells; (c) U937 cells.

### DETAILED DESCRIPTION OF THE INVENTION

The invention will be further described by the following embodiments. It will be appreciated that the following embodiments are used to illustrate the present invention not to limit the scope of it.

The cell lines used in the following embodiments are from shanghai institute of hematology, including: Kasumi-1 cell line which is from AML subtype with t (8; 21) (q22; q22), (AML1/ETO), and NB4-R1 cell line from AML subtype with t (15; 17) (q22:q12), (PML-RARα) and resistant to retinoic acid, HL60 cell line from AML-M2 subtype, and U937 cell line from AML-M5 subtype.

### EXAMPLE 1

### The Preparation of Artemisinin Derivative Diarteethe Amine Maleate

This embodiment starts from the known compound hydroxy diarteether (Reference: Ying Li et al, Acta Pharmaceutica Sinica, 1981,16:429-439). First we manufacture its tosylester, and then let it react with ammonia water in solution dimethylformamide, which produces diarteethe amine. The reaction route is as follow:

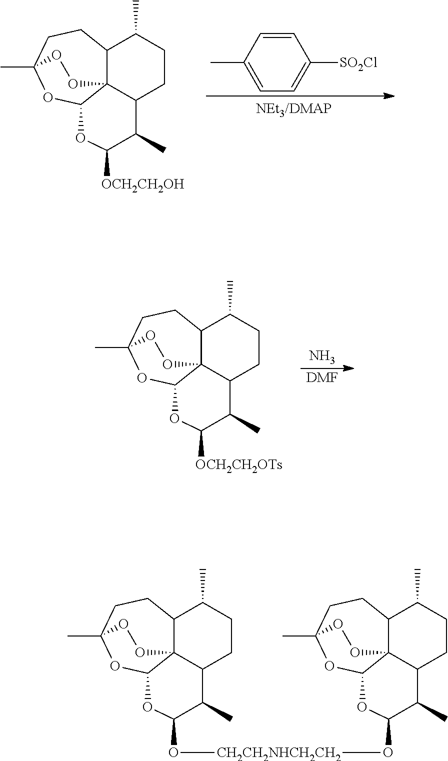

Afterwards, make the diarteethe amine into its maleate.

The concrete operations are as follows:

Dissolve the hydroxy diarteethe tosylester (1.54 g) in dimethylformamide, then add ammonia water (0.5 ml), and stir it for 20 h at 40-50° C. When the materials spots are almost disappeared in thin layer chromatography (TCL), pour the reaction solution into the ice water, extract repeatedly with ethyl acetate, combining organic phases, washing in saturated brine, drying with anhydrous glauber's salt. Clearing off solvent by reduced pressure distillation, and passing the residue through column chromatography (silica, the eluent is the mixture of ethyl acetate, petroleum ether and triethylamine, and the concentration of gradient elution is 1/19/1 1/10/1,v/v/v). Herein we get 0.4 g buff oily product, and the yield coefficient is 40%. Dissolving the oily product with a little ethyl acetate, adding the solution of maleic acid and ethyl acetate in drops until it turns out to be weak acidic, after which it solidifies. Filtering the maleate, ethanol and petroleum ether recrystalizing, herein we get white crystals. Melting point: 140˜142° C. ¹HNMR (free alkali, 300 MHz, CDCL₃) δ:5.40 (s, 2H), 4.82 (d, J=3.3 Hz, 2H), 3.97 (m, 2H), 3.56 (m, 2H), 2.84 (m, 4H), 1.43 (s, 6H), 0.95 (d, J =6.0 Hz, 6H), 0.90 (d, J=7.2Hz, 6H). Mass Spectrometry (free alkali C₃₄H₅₅NO₁₀): m/z 638 (M+1)⁺. Elemental analysis (maleate, C₃₈H₅₉NO₁₄): the calculated value: C 60.53, H7.89, N1.86; measured value: C 60.72, H 8.00, N 1.73.

According to the data above, it is certain that the structure formula of diarteethe amine maleate is as follow:

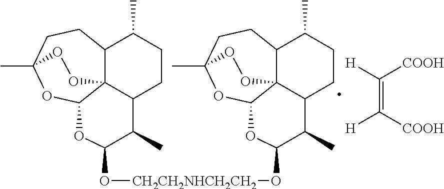

### EXAMPLE 2

### The Inhibition Test of SM1044 to Leukemic Cells

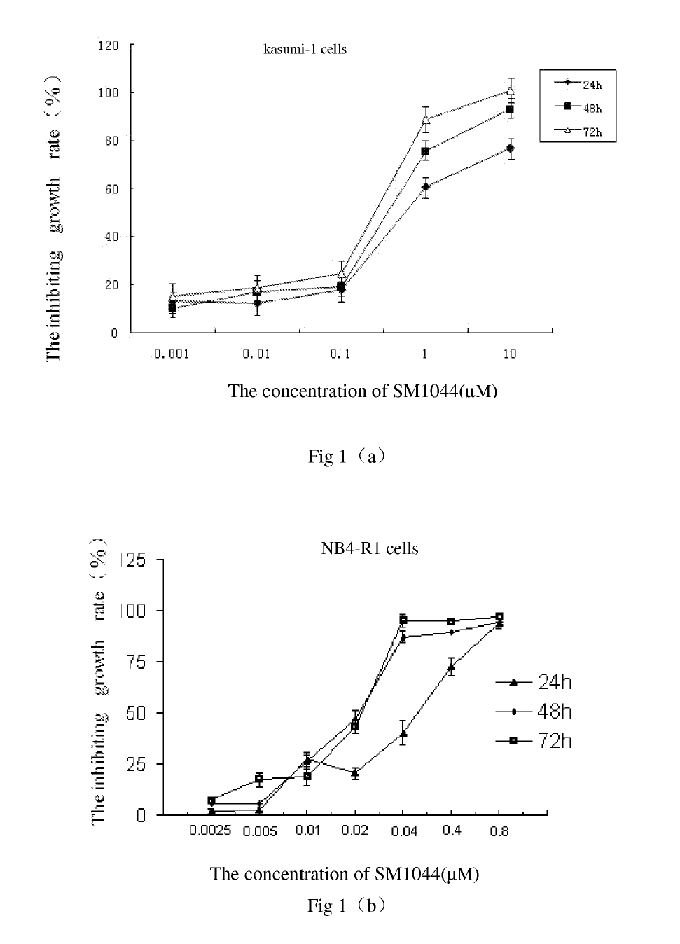

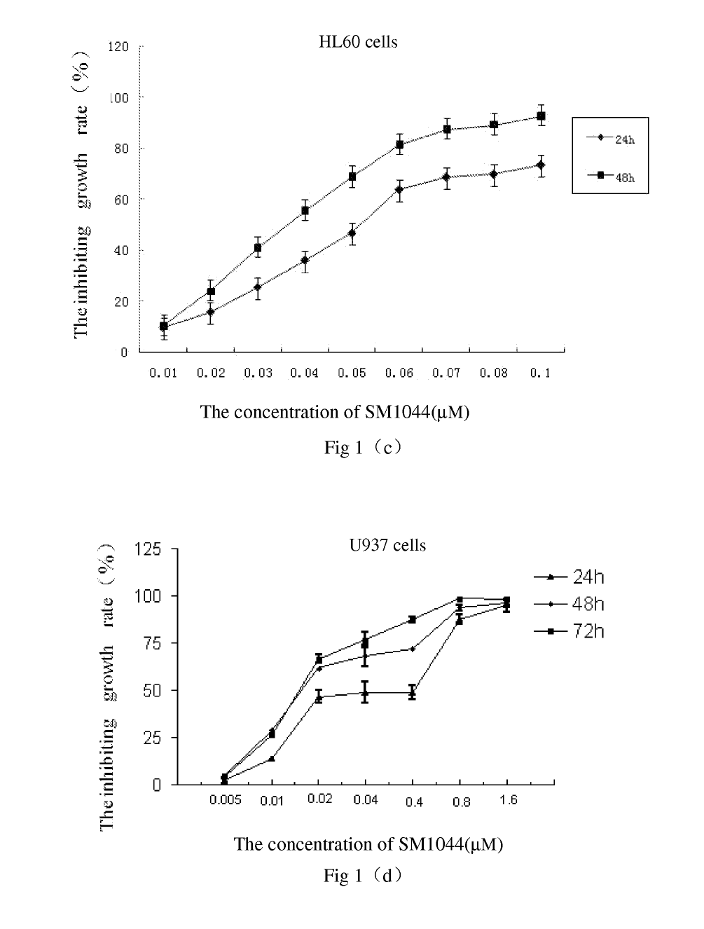

First dissolve SM1044 in triple distilled water, concentration: 1 mg/ml, then choose typical AML cell lines such as Kasumi-1, NB4-R1, HL60 and U937 cells to perform the experiments. Plate 2.5×10⁵ cells in dishes, respectively, blank group, control group without SM1044, and groups with SM1044 in various concentrations, and 3 wells for each concentration. Add MTT (5 mg/ml) to each well after incubating for a certain period, and incubate the plate for further 4 h. Centrifuge and aspirate 180 μl supernatant. Add 180 μl DMSO to each well, and place the plate a shaking table for 15 min. Measure the absorbance(A) at 570 nm using a microplate reader, and calculate the half maximal inhibitory concentration (IC₅₀). It turns out that the four AML cells are all sensitive to SM1044, and at 48 h, IC₅₀ are respectively 0.17 μM (Kasumi-1 cells, FIG. 1(a)), 0.021 μM (NB4-R1 cells, FIG. 1(b)), 0.04 μM (HL60 cells, FIG. 1(c)), 0.02 μM (U937cells, FIG. 1(d)), which indicates that SM1044 is able to inhibit the proliferation of leukemic cells.

### EXAMPLE 3

### SM1044 Inducing the Apoptosis of Leukemic Cells

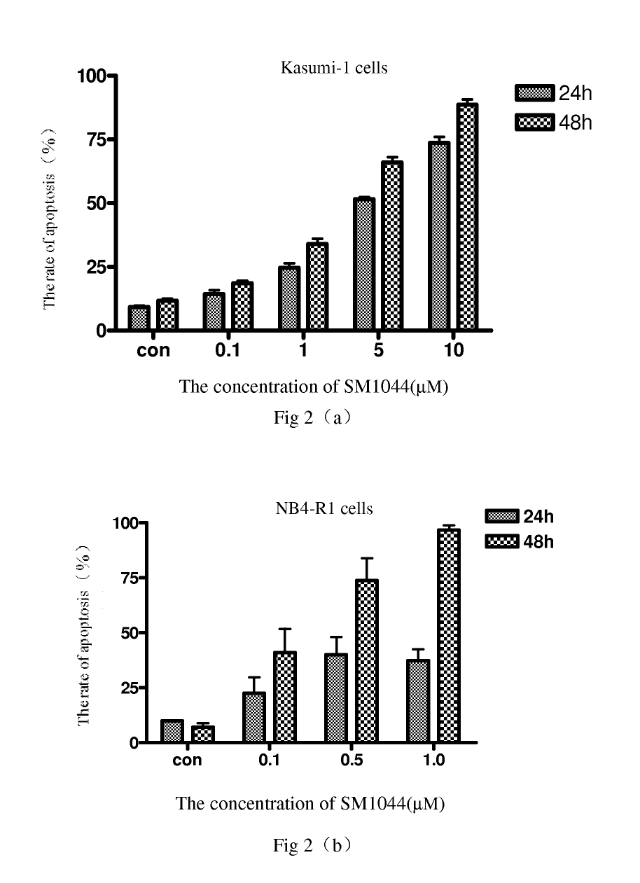

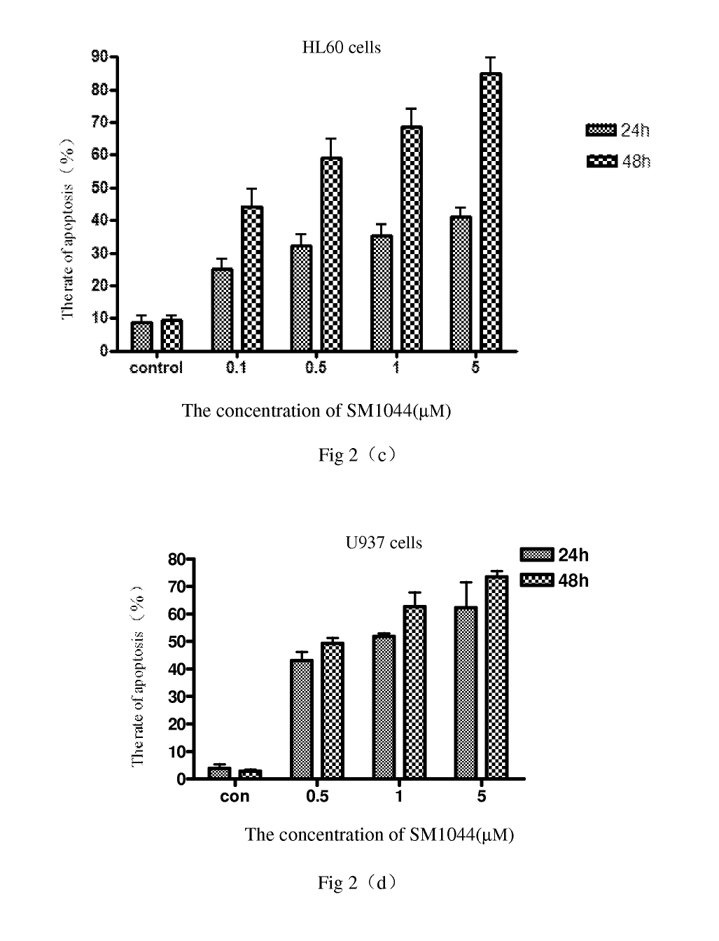

Respectively plate 3×10⁵˜5×10⁵ cells/m1 of Kasumi-1, NB4-R1, HL60 and U937cells in dishes. Setting control group (0 μM) and groups with SM1044 in various concentrations, incubate for 24 h and 48 h. Harvest cells and wash cells with pre-cooling phosphate buffer twice. Resuspend the cells in 200 μL binding buffer, and add 5 μL Annexin V-FITC and 5 μL propidium iodide (PI), gently mixing, incubating for 15 min at room temperature in the dark, analyzing by flow cytometry in 1 hour. The results show that SM1044 can induce apoptosis of Kasumi-1 (FIG. 2(a)), NB4-R1 (FIG. 2(b)), HL60 (FIG. 2(c)) and U937 cells (FIG. 2(d)) after 24-48 hours in various concentrations. It is suggested that SM1044 can induce the apoptosis of leukemic cells, and the higher the concentration is and the longer the time mainteins, the higher the apoptosis rate is.

### EXAMPLE 4

### SM1044 Inducing the Loss of Mitochondrial Membrane Potential of the AML Cells

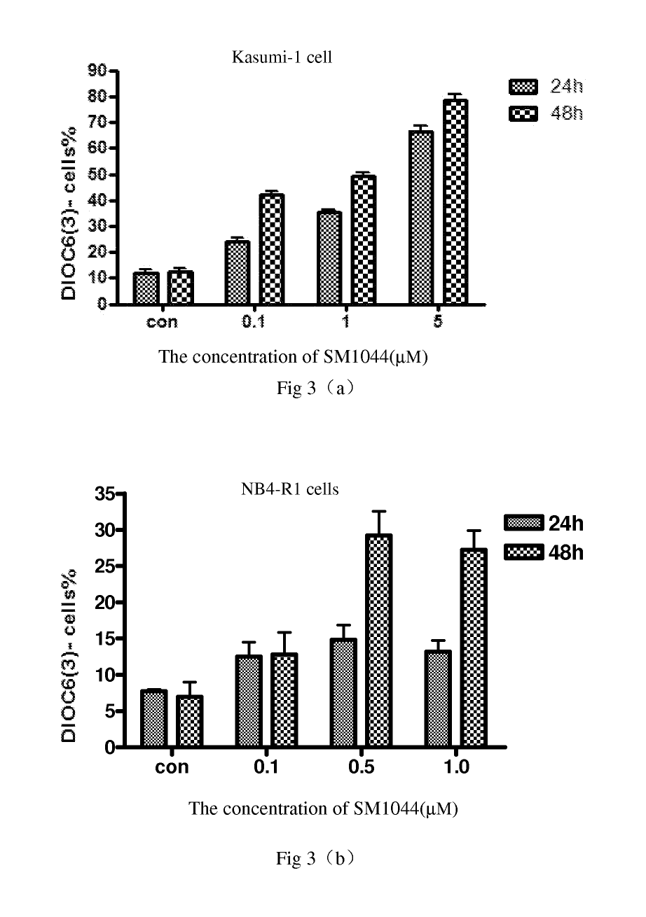

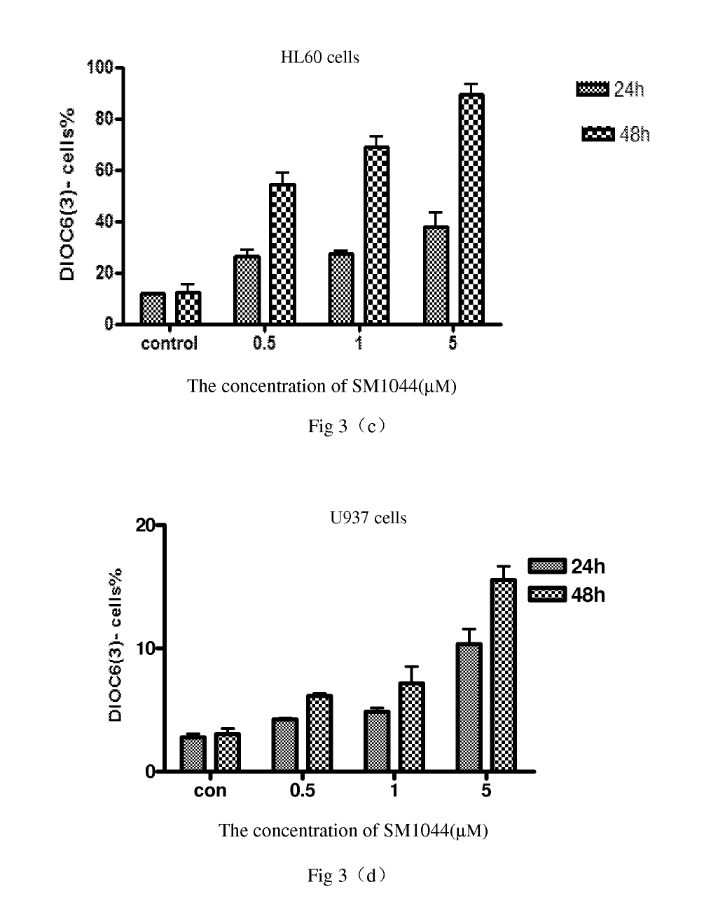

Respectively plate 3×10⁵˜5×10⁵ cells/ml of Kasumi-1, NB4-R1, HL60 and U937cells in dishes. Setting control group (0 μM) and groups with SM1044 in various concentrations, incubate for 24 h and 48 h. Add 20 nM DiOC6(3) and incubate for 15 min at 37° C. in the dark. Wash cells with PBS twice, and resuspend the cells in 100 μLPBS, analyzing by flow cytometry. The cells which are DiOC6(3) negative are those that lose mitochondrial membrane potential. The results show that SM1044 induces the loss of mitochondrial membrane potential of Kasumi-1 (FIG. 3(a) and table 1), NB4-R1 (FIG. 3(b) and table 2), HL60 (FIG. 3(c) and table 3) and U937 (FIG. 3(d) and table 4), which is time-dependent and concentration-dependent. There are at least two broad pathways that lead to Apoptosis, an “Extrinsic” and an “Intrinsic” Pathway, and the loss of mitochondrial membrane potential is very closely related to intrinsic pathway. It is suggested that SM1044 induces apoptosis through intrinsic pathway.

TABLE 1
The percentage of SM1044 inducing
DiOC₆(3) negtive Kasumi-1 cells

| | Con | 0.1 μM | 1 μM | 5 μM |
|---|---|---|---|---|
| 24 h | 10.3% | 24.1% | 33.8% | 63.2% |
| 48 h | 10.2% | 44.9% | 47.1% | 79.2% |

TABLE 2
The percentage of SM1044 inducing
DiOC₆(3) negative NB4-R1 cells

| | Con | 0.1 μM | 1 μM | 5 μM |
|---|---|---|---|---|
| 24 h | 7.7% | 13.9% | 17.1% | 15.0% |
| 48 h | 8.8% | 16.3% | 33.0% | 30.0% |

TABLE 2
The percentage of SM1044 inducing
DiOC₆(3) negative HL-60 cells

| | Con | 0.1 μM | 1 μM | 5 μM |
|---|---|---|---|---|
| 24 h | 10.9% | 23.8% | 25.6% | 48.5% |
| 48 h | 18% | 61.7% | 77.1% | 81.1% |

TABLE 2
The percentage of SM1044 inducing DiOC₆(3) negative U937 cells

| | Con | 0.1 μM | 1 μM | 5 μM |
|---|---|---|---|---|
| 24 h | 3.1% | 4.3% | 5.2% | 9.4% |
| 48 h | 2.5% | 5.9% | 5.6% | 16.2% |

### EXAMPLE 5

### SM1044 Inducing the Formation of Apoptosis-Related Proteins in the AML Cells

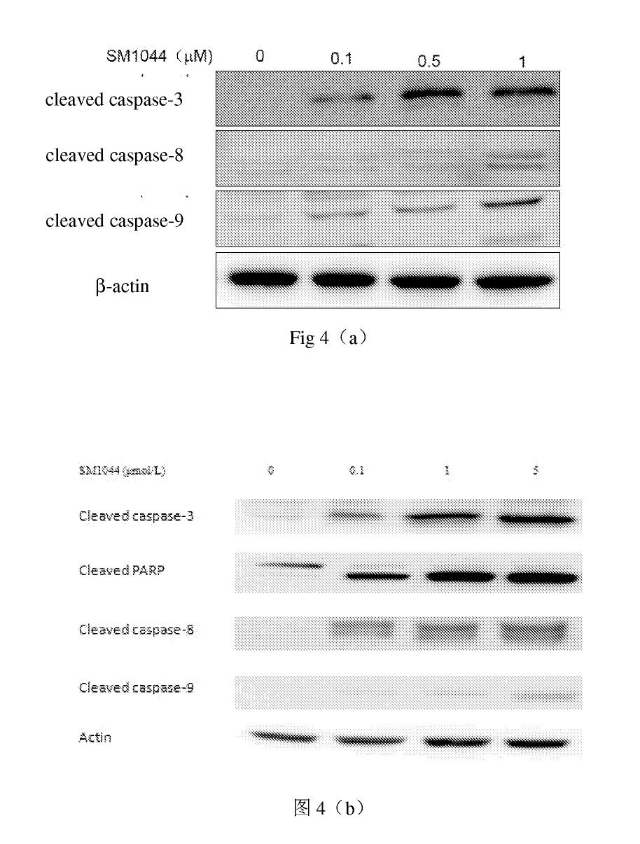

Respectively plate 3×10⁵˜5×10⁵ cells/ml of NB4-R1, HL60 and U937 cells in dishes. Setting control group (0 μM) and groups with SM1044 in various concentrations, and incubating for 24 hours. Extracting whole protein of the cells, detect the quantitive variation of antiapoptotic related proteins (PARP, caspase-3, caspase-8, caspase-9) by antibodies through western blot. Caspase family plays an important role in mediating apoptosis. There are at least two broad pathways that lead to Apoptosis. One is extracellular signals activating intracellular caspase-8, etc, the other is mitochondria-derived activators of caspases activating caspase-9, etc. Therefore the activaigted caspases activate apoptotic excutants—caspase3 to induce apoptosis, and PARP is the substrate of caspase-3. The results indicate that activated apoptosis-related proteins such as caspase-3, caspase-8 and/or caspase-9 increase after being treated with SM1044 in NB4-R1 (FIG. 4(a)), HL60 (FIG. 4(b)) and U937 (FIG. 4(c)), which suggests that SM1044 is able to induce apoptosis through both extrinsic and intrinsic pathway.

### EXAMPLE 6

### SM1044 Inducing Degradation of the Kasumi-1 Specific AML1-ETO Fusion Protein

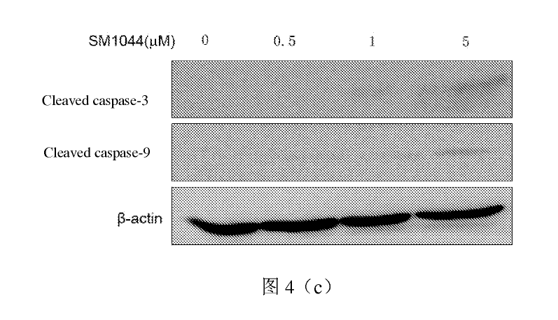

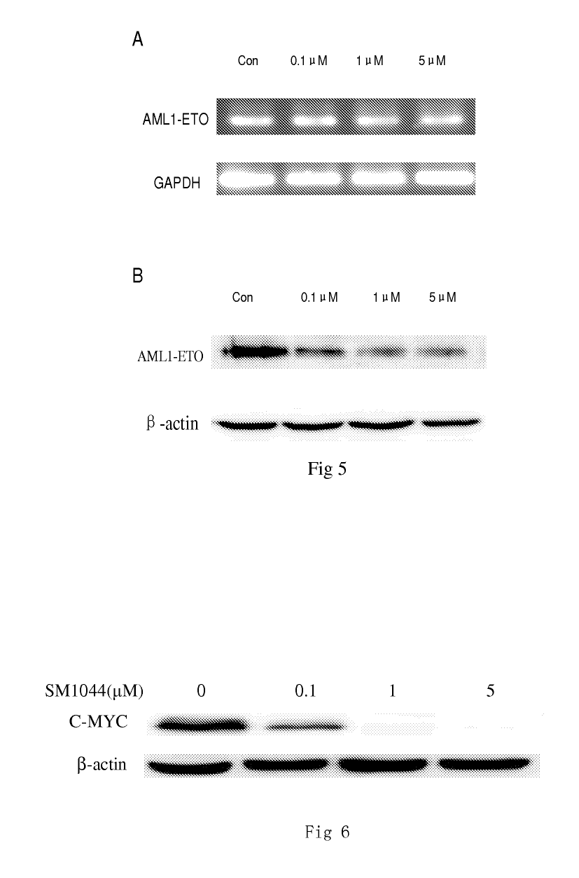

Plate 1×10⁷ Kasumi-1 cells with 20 mL medium in dish. Setting control group (0 μM) and groups with SM1044 in various concentrations, and incubating for 24 hours. Extract the whole RNA, and detect the variation of fusion gene at the mRNA level by RT-PCR (FIG. 5A). It shows that SM1044 has no effect on the transcription of fusion gene. Plate 5×10⁵ Kasumi-1 cells in a dish. Setting control group (0 μM) and groups with SM1044 in various concentrations, and incubating for 24 hours. Extracting whole protein of the cells, detect the variation of AML1-ETO fusion protein by ETO antibody through western blot (FIG. 5B). It shows that SM1044 can degradate AML1-ETO fusion protein. When the concentration of SM1044 is 1 μM/L, it can induce the degradation of AML1-ETO fusion protein. It can be concluded that SM1044 doesn't effect the transcription of AML1-ETO fusion gene, but can induce the degradation of the fusion protein. As the typical fusion gene of AML-M2b, AML1-ETO fusion protein plays an important role in the occurrence of leukemia, which can be a target for medical treatments. The present embodiment obviously shows that SM1044 can induce the degradation of AML1-ETO fusion protein, which manifests AML1-ETO fusion protein could be an intracellular target for SM1044.

### EXAMPLE 7

### SM1044 Inhibiting the Expression of Oncogene c-myc in HL60 Cells

Plate 3×10⁵/mL HL60 cells in dishes. Setting control group (0 μM) and groups with SM1044 in various concentrations, and incubating for 24 hours. Extracting whole protein of the cells, detect the variation of expression of oncogene c-myc by c-myc antibody through western blot. Over-expression of oncogene c-myc is a character of HL60 cells. When the concentration of SM1044 is above 1 μM/L, it can completely inhibit the expression of oncogene c-myc (FIG. 6), which indicates that SM1044 can inhibit the expression of oncogene c-myc.

### EXAMPLE 8

### SM1044 Blocking the Cell Cycle of AML Cells

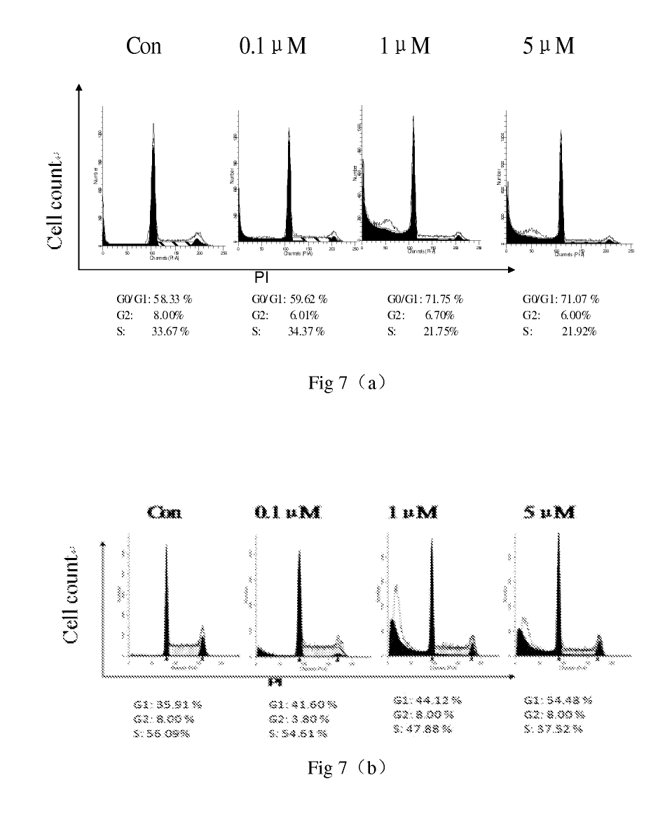

Plate 5×10⁵/mL Kasumi-1 cells and HL60 cells respectively in dishes. Setting control group (0 μM) and groups with SM1044 in various concentrations, and incubating for 24 hours (kasumi-1 cells) or 12 hours (HL60 cells). Harvest the cells, wash cells with PBS twice, and add 70% ice cold ethanol overnight to fix the cells. Wash cells with PBS, and resuspend the cells in PBS with 10 mg/ml RNase, incubating at 37° C. for 30 min. Add 50 μg/ml propidium iodide (PI), analyzing the distribution of DNA contents by flow cytometry in 1 hour. The results show that Kasumi-1 (FIG. 7(a) and Table 5) and HL60 cells (FIG. 7(b) and Table. 6) treated with SM1044 are blocked in G₀/G₁ phase, the cells in G₀/G₁ phase increasing. It is suggested that SM1044 can block cell cycles and stop growing.

TABLE 5
The effect of SM1044 to the cell cycle of Kasumi-1 cells

| | Con | 0.1 μM | 1 μM | 5 μM |
|---|---|---|---|---|
| G₀/G₁ | 58.33% | 59.62% | 71.75% | 71.07% |
| G₂/M | 8.00% | 6.01% | 6.70% | 6.00% |
| S | 33.67% | 34.37% | 21.75% | 21.92% |

TABLE 6
The effect of SM1044 to the cell cycle of HL60 cells

| | Con | 0.1 μM | 1 μM | 5 μM |
|---|---|---|---|---|
| G₀/G₁ | 35.91% | 41.60% | 44.12% | 54.48% |
| G₂/M | 8.00% | 3.80% | 8.00% | 8.00% |
| S | 56.09% | 54.61% | 47.88% | 37.52% |

### EXAMPLE 9

### SM1044 Can Inhibit the Growth of Transplanted Tumor with AML Cells in Mice

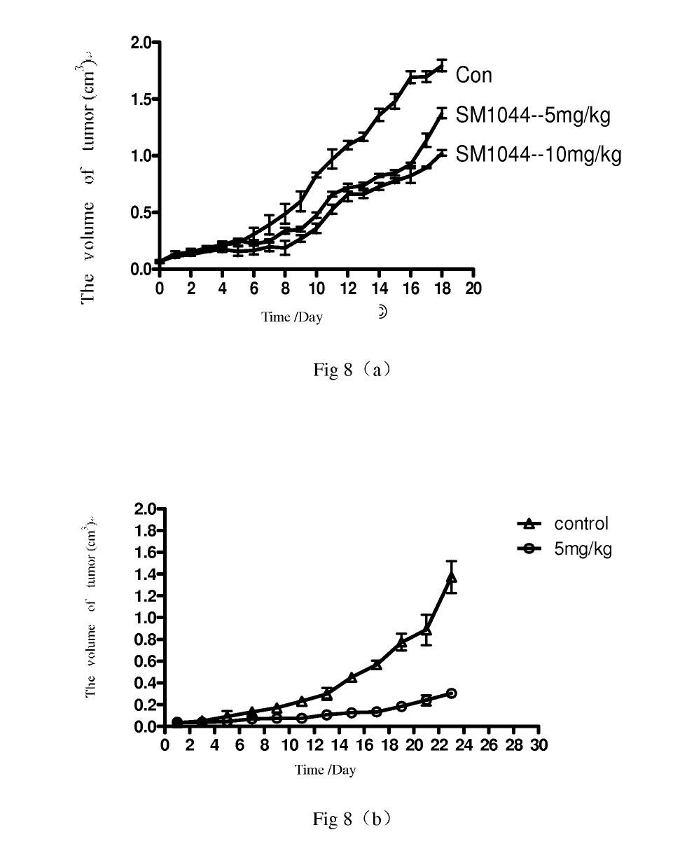

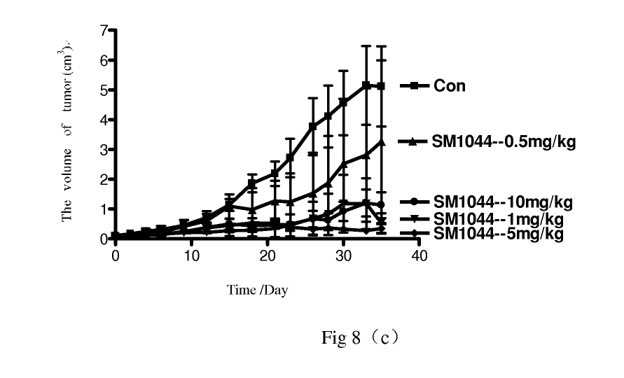

Developing mouse transplanted tumor models with acute myeloid leukemic cell lines such as Kasumi-1, HL60 and U937 cells, inject 1×10⁷ leukemic cells under the skin. When the tumors are 5 mm in diameter, divide the mice into two groups, control group(con) and groups with SM1044 in various concentrations. Mice intraperitoneally infused with 0.125 ml (kasumi-1 and H160 cells) or 0.1 ml (U937 cells) SM1044 per day, while mice in control groups get equivalent saline, for a course of 18-35 d, measuring the tumors everyday. The results show that, in mice transplanted tumor models with Kasumi-1 (FIG. 8(a)), HL60 (FIG. 8(b)) and U937 (FIG. 8(c)) cells, compared with control groups, tumors in groups with SM1044 are obviously little than controls, which indicating that SM1044 can inhibit the proliferation of cancer cells in mice.

Artemisinin, a sesquiterpene lactone containing an unusual peroxide bridge, isolated from the plant Artemisia annua, and its derivatives includes artesunate, artemether and dihydroartemisinin, etc. Nowadays, Artemisinins have a role in first-line therapy for malaria worldwide. Although, in recent 20 years, artemisinin is undergoing early research and testing for the treatment of cancer, which demonstrates that artemisinin has significant anticancer effects in vitro, the effects in vivo are not that good. We are trying to study a novel kind of artemisinin-derivatives—diarteether amine and its hydrosoluble salts, in order to enhance the effect of anti-cancer. The present invention utilizes a novel hydrosoluble artemisinin derivative—diarteethe amine maleate, and analyzes the the inhibitory curve of AML cells, etc, which shows that SM1044 can inhibit the proliferation of leukemic cells, and induce apoptosis, with time and dosage denpendence. SM1044 can be used for the preparation of anti-leukemia medicines, especially for treatment of acute leukemia and, what's more, for the treatment of acute myeloid leukemia.

It should be appreciated that embodiments mentioned above are used to illustrate the present invention not to limit the scope of it. Though the selected embodiments have been illustrated in detail, the technicians in this field should noted that modifications or equivalent replacements of the technical proposals, such as replacing maleate with other hydrosoluble salts of diarteether amine, could achieve the same effect. The artemisinin-derivatives-diarteether amine mentioned in the present invention, is able to induce apoptosis of leukemic cells, can inhibit cancer cells in short time, at low dosage, with less side effect, which means a lot to the treatment of leukemia.

1-4. (canceled)

5. A method of treating leukemia in a subject in need, comprising administering to said subject a composition comprising an effective amount of an artemisinin-derivative, or pharmaceutical salts thereof, wherein said artemisinin-derivative is a diarteethe amine with a structure formula shown as follows:

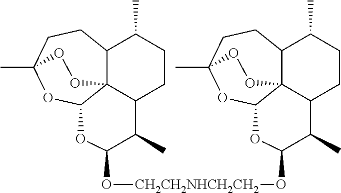

6. The method of claim 5, wherein said leukemia is an acute leukemia.

7. The method of claim 6, wherein said acute leukemia is an acute myeloid leukemia (ALM).

8. The method of claim 7, wherein said acute myelocytic leukemia is selected from the group consisting of AML with t(8;22)(q22;q22)(AML/ETO), AML with t(15;17)(q22;q21), (PML-RARα) in AML with recurrent genetic abnormalities, AML-M2, and AML-M5.

9. A method of preparing a medicament for treating leukemia, comprising:
a) converting hydroxyl diarteethe to tosylester;
b) reacting tosylester in solution dimethylformamide and with ammonia water; and
c) producing diarteethe amine and its maleate
wherein said diarteethe amine has the structural formula shown as follows:

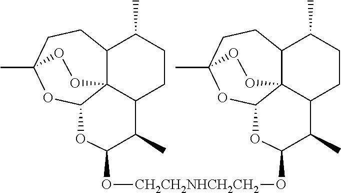

10. The method of claim 9, wherein said diarteethe amine is used for treating an acute leukemia.

11. The method of claim 10, wherein said acute leukemia is an acute myeloid leukemia (ALM).

12. The method of claim 11, wherein said acute myelocytic leukemia is selected from the group consisting of AML with t(8;22)(q22;q22)(AML/ETO), AML with t(15;17)(q22;q21), (PML-RARα) in AML with recurrent genetic abnormalities, AML-M2, and AML-M5.
